(19) United States
(12) Patent Application Publication
(10) Pub. No.: US 2009/0054702 A1
(43) Pub. Date: Feb. 26, 2009

(54) HYDROGENATION PROCESS

(76) Inventor: Joseph Broun Powell, Houston, TX (US)

Correspondence Address: SHELL OIL COMPANY, P O BOX 2463, HOUSTON, TX, 772522463, US

(21) Appl. No.: 12/131,638

(22) Filed: Jun. 2, 2008

Related U.S. Application Data
(60) Provisional application No. 60/941,918, filed on Jun. 4, 2007.

(51) Int. Cl. C07C 31/18

(52) U.S. Cl. 568/863

(57) ABSTRACT

The present invention provides a process for hydrogenating an aldehyde. In one aspect, the invention is directed to a process of hydrogenating an aldehyde with a catalyst comprising a Group VIII metal, where the catalyst is complexed with carbon monoxide, at a temperature of at least 120° C. In another aspect, the invention is directed to a process of hydrogenating an aldehyde by contacting a feed comprising the aldehyde with a Group VIII metal catalyst and hydrogen in the presence of carbon monoxide at a temperature of at most 90° C. and subsequently contacting the feed and catalyst with hydrogen at a temperature of at least 120° C.

This application claims the benefit of U.S. Provisional Application No. 60/941,918 filed Jun. 4, 2007, the entire disclosure of which is hereby incorporated by reference.

### FIELD OF THE INVENTION

The present invention relates to a hydrogenation process. In particular, the present invention relates to a process for hydrogenating an aldehyde.

### BACKGROUND OF THE INVENTION

1,3-propanediol (“PDO”) is an industrially important chemical. PDO may be used as a monomer unit to form polymers such as poly(trimethylene terephthalate) that are used in the production of textiles and carpets. PDO is also useful as an engine coolant, particularly in cooling systems that require coolants having low conductivity and low corrosivity.

PDO may be prepared in a two-step process in which ethylene oxide is first hydroformylated in an organic solution in the presence of a metal catalyst such as cobalt or rhodium carbonyl to form 3-hydroxypropionaldehyde. The hydroformylation is conducted in the presence of carbon monoxide and hydrogen, typically present in the hydroformylation reaction system as syngas introduced into the reaction system at relatively high pressure. The carbon monoxide is used in combination with a reactive metal species such as rhodium or cobalt to form the metal carbonyl hydroformylation catalyst. After hydroformylation, the 3-hydroxypropionaldehyde is extracted from the organic solution with an aqueous solution under pressure, typically under a carbon monoxide partial pressure sufficient to minimize extraction of the metal carbonyl hydroformylation catalyst into the aqueous extractant. In the second step, the aqueous extract of 3-hydroxypropionaldehyde is hydrogenated in the presence of a hydrogenation catalyst to form PDO.

Ideally, the aqueous 3-hydroxypropionaldehyde hydroformylation extract could be routed directly to the hydrogenation reactor. However, carbon monoxide dissolved in the aqueous 3-hydroxypropionaldehyde hydroformylation extract is a poison for hydrogenation catalysts containing Group VIII metals at temperatures effective to convert 3-hydroxypropionaldehyde to PDO without producing substantial amounts of byproducts. Specifically, hydrogenation of 3-hydroxypropionaldehyde is typically initially conducted at temperatures of at most 90° C. to avoid significant formation of undesired byproducts. Carbon monoxide is an irreversible poison for hydrogenation catalysts utilizing a Group VIII metal as the active hydrogenation catalyst metal at temperatures of 120° C. or less, and carbon monoxide may severely suppress catalytic activity at temperatures required to selectively hydrogenate the aldehyde.

In order to prevent poisoning the hydrogenation catalyst, carbon monoxide is typically removed from the aqueous hydroformylation product prior to hydrogenation. Typically, removal of the carbon monoxide from the aqueous aldehyde hydroformylation product entails depressurizing the product, stripping the carbon monoxide from the depressurized product mixture with an inert gas, and repressurizing the product mixture with hydrogen prior to hydrogenating the aldehyde. Significant expense in equipment, material, energy, and time is required to remove the carbon monoxide from the aqueous hydroformylation product prior to hydrogenation by such depressurizing, stripping, and repressurizing. Further, some aldehydes, such as 3-hydroxypropionaldehyde, may attack the internal structure of the repressurizing pump. It would be useful, therefore, to be able to effect hydrogenation of at least a majority of an aldehyde such as 3-hydroxypropionaldehyde with a Group VIII metal catalyst in the presence of carbon monoxide without forming a substantial amount of byproducts.

U.S. Pat. No. 5,786,524 discloses a process for hydrogenating an aqueous extract of a hydroformylation reaction mixture containing 3-hydroxypropionaldehyde. The hydrogenation is effected in one stage or in two or more sequential temperature stages, where a preferred hydrogenation process hydrogenates the aqueous 3-hydroxypropionaldehyde hydroformylation extract in two or more hydrogenation stages where the first hydrogenation stage has a temperature of about 50° C. to about 130° C., the second hydrogenation stage has a temperature higher than the first hydrogenation stage and within the range of about 70° C. to about 155° C. The aqueous 3-hydroxypropionaldehyde extract may be oxidized and passed through an acid ion exchange resin bed prior to hydrogenation. As shown in U.S. Pat. No. 5,786,524, this is effective to separate carbon monoxide and residual hydroformylation catalyst metals from the aqueous 3-hydroxypropionaldehyde hydroformylation extract. The process disclosed in U.S. Pat. No. 5,786,524 is not effective to hydrogenate at least a majority of an aldehyde such as 3-hydroxypropionaldehyde with a Group VIII catalyst in the presence of carbon monoxide without forming a substantial amount of byproducts.

### SUMMARY OF THE INVENTION

In an aspect, the present invention is directed to a process for hydrogenating an aldehyde comprising contacting a feed comprising an aldehyde with hydrogen and with a catalyst at a temperature of at least 120° C., where the catalyst comprises a Group VIII metal, or a compound containing a Group VIII metal, and where the Group VIII metal or Group VIII metal compound is complexed with carbon monoxide.

In an aspect, the present invention is directed to a process for hydrogenating an aldehyde in the presence of carbon monoxide, comprising: (a) contacting a feed comprising an aldehyde with hydrogen and a catalyst comprising a Group VIII metal or a compound containing a Group VIII metal at a temperature up to 90° C., or from 20° C. to 85° C., or from 30° C. to 80° C. in the presence of carbon monoxide; and (b) subsequent to step (a), contacting the feed and catalyst with hydrogen at a temperature of at least 120° C., or from 120° C. to 180° C., to produce a hydrogenation product.

In an aspect, the present invention is directed to a process for producing 1,3-propanediol, comprising: (a) providing an aqueous feed comprising 3-hydroxypropionaldehyde; (b) contacting the feed with hydrogen and a catalyst comprising a Group VIII metal or a compound containing a Group VIII metal at a temperature of up to 90° C., or from 30° C. to 85° C., or from 40° C. to 80° C. in the presence of carbon monoxide; and (c) subsequent to step (b), contacting the feed and catalyst with hydrogen at a temperature of from 120° C. to 180° C. to produce a hydrogenation product mixture containing 1,3-propanediol.

### BRIEF DESCRIPTION OF THE DRAWINGS

One or more systems useful for practicing one or more embodiments of the invention are illustrated, by way of example only, with reference to the following drawings:

FIG. 1 is a schematic illustrating a system useful in the process of hydrogenating an aldehyde utilizing a hydrogenation catalyst comprising a Group VIII metal with a single hydrogenation reactor.

FIG. 2 is a schematic illustrating a system useful in the process of hydrogenating an aldehyde utilizing a hydrogenation catalyst comprising a Group VIII metal with more than one hydrogenation reactor.

### DETAILED DESCRIPTION OF THE INVENTION

The present invention provides a process for hydrogenating an aldehyde utilizing a catalyst containing a Group VIII metal or a Group VIII metal compound (hereafter collectively referred to as “Group VIII metal catalysts”) in the presence of carbon monoxide without forming a substantial amount of byproducts, for example, there may be less than 1% molar loss of the aldehyde to byproducts, or less than 0.1% molar loss of the aldehyde to byproducts. It has been found that carbon monoxide is associatively adsorbed on Group VIII metal catalysts during hydrogenation at temperatures of 90° C. or below, however, the associatively adsorbed carbon monoxide disproportionates to surface carbon and carbon dioxide during hydrogenation at temperatures of at least 120° C. The surface carbon, once formed, is readily hydrogenated to methane, freeing the surface of the Group VIII metal catalyst from deactivating carbon compounds.

In an aspect, therefore, the present invention is directed to hydrogenating an aldehyde by contacting a feed containing the aldehyde with hydrogen and with a catalyst at a temperature of at least 120° C., where the catalyst is comprised of a Group VIII metal and/or a Group VIII metal compound, and where the catalyst is complexed with carbon monoxide. Under these hydrogenation conditions, the carbon monoxide on the catalyst disproportionates to surface carbon and carbon dioxide, where the surface carbon is hydrogenated to methane. The catalyst is freed from deactivating carbon monoxide and actively catalyzes hydrogenation of the aldehyde.

In another aspect of the invention, an aqueous solution of the aldehyde may be hydrogenated in the presence of carbon monoxide and a Group VIII metal catalyst in at least two stages, where the initial hydrogenation stage is a low temperature hydrogenation conducted at one or more temperatures of up to 90° C. and a subsequent hydrogenation stage is a high temperature hydrogenation conducted at one or more temperatures of at least 120° C. One advantage of initially hydrogenating the aldehyde at a temperature of up to 90° C. is that hydrogenation at such a relatively low temperature may limit the formation of undesired byproducts which are observed when high concentrations of aldehyde are hydrogenated at high temperature. Carbon monoxide may adsorb to the Group VIII metal catalyst in the initial hydrogenation stage, but is disproportionated and removed from the catalyst in the following high temperature hydrogen step. The hydrogenation is subsequently continued at one or more temperatures of at least about 120° C. to convert most, if not all, of the remaining aldehyde. Advantages of continuing hydrogenating at a temperature of at least 120° C. are that 1) most, if not all, the remaining aldehyde may be converted; 2) some byproducts formed in hydroformylation or other processing steps, or in the initial hydrogenation at temperatures up to 90° C., such as acetals, may be converted to the desired product; and 3) the catalyst may be regenerated by removal of carbon monoxide. Carbon monoxide induced catalyst poisoning that may have occurred while hydrogenating at temperatures of at most 90° C. may be reversed by conducting the hydrogenation at temperatures of at least 120° C., thereby regenerating the catalyst's activity. The Group VIII metal catalyst freed of deactivating carbon compounds may continue to be utilized in a high temperature hydrogenation or may be re-used to hydrogenate the aldehyde at lower temperatures, e.g. below 90° C. Limited amounts of byproducts are formed in the hydrogenation process since the initial hydrogenation may be conducted at low temperatures despite the presence of carbon monoxide.

In an embodiment of the present invention, an aldehyde formed in the presence of carbon monoxide may be directly hydrogenated without removing the aldehyde from the presence of the carbon monoxide by using a multiple temperature stage hydrogenation process, where the initial hydrogenation temperature is at most 90° C. and at least one subsequent hydrogenation temperature is at least 120° C. The process is especially advantageous for direct hydrogenation of a hydroformylation reaction mixture, or an extract thereof, without separating carbon monoxide from the hydroformylation reaction mixture.

In the process of the invention, the feed comprises an aldehyde. The aldehyde may be any aldehyde that may be hydrogenated to an alcohol, diol, triol, or polyol. In one embodiment, the aldehyde may be a straight or branched chain aliphatic aldehyde. In an embodiment, the straight or branched chain aliphatic aldehyde may comprise at most 8 carbon atoms, or may contain from 2 to 6 carbon atoms.

In an embodiment, the aldehyde is a 3-hydroxyaldehyde, i.e. a compound of the general formula

R₂C(OH)—C(R)₂—CH═O

wherein each R independently may be a hydrogen atom or may jointly) be a hydrocarbon group that is substituted or unsubstituted, and/or aliphatic or aromatic. Each group R may independently vary in size, for instance, from 1 to 20 carbon atoms, preferably from 1 to 10 carbon atoms. In addition, they may bear one or more substituents selected from hydroxyl, alkoxy, carbonyl, carboxy, amino, cyano, cyanto, mercapto, phosphino, phosphonyl, and or silyl groups, and/or one or more halogen atoms. Preferred 3-hydroxyaldehydes are those having in total from 3 to 12 carbon atoms, and more preferably from 3 to 8 carbon atoms. Most preferably the 3-hydroxyaldehyde is 3-hydroxypropionaldehyde, i.e. wherein each R is a hydrogen atom.

The feed may be a solution containing the aldehyde, preferably 3-hydroxypropionaldehyde, where the solution may be an aqueous solution comprising at least 50 wt. %, or at least 70 wt. %, or at least 90 wt. %, or at least 95 wt. % water based on the weight of the aqueous feed solution, or an organic solution comprising at least 50 wt. %, or at least 70 wt. %, or at least 90 wt. %, or at least 95 wt. % of one or more organic species such as an organic solvent based on the weight of the organic feed solution. The aldehyde is preferably soluble in the feed solution, e.g. if the feed solution is aqueous the aldehyde is preferably soluble in the aqueous feed solution, and if the feed solution is organic the aldehyde is preferably soluble in the organic feed solution. A vapor feed containing aldehyde may be employed. In an embodiment, the aldehyde may be subject to dehydration under conditions for hydrogenating the aldehyde, and the feed solution may contain at least 1 wt. %, or at least 5 wt. %, or at least 20 wt. %, or at least 70 wt. % of water, where the water may inhibit dehydration of the aldehyde under hydrogenation conditions.

The initial feed solution may contain at least 0.1 wt. % of the aldehyde, at least 0.2 wt. % of the aldehyde, at least 0.3 wt. % of the aldehyde, at least 0.5 wt. % of the aldehyde, or at least 1 wt. % of the aldehyde based on the liquid weight of the feed solution. The initial feed solution may contain at most 15 wt. % of the aldehyde, at most 12 wt. % of the aldehyde, at most 10 wt. % of the aldehyde, or at most 8 wt. % of the aldehyde based on the liquid weight of the feed solution. The initial feed solution may contain from 0.1 wt. % to 15 wt. % of the aldehyde, from 0.2 wt. % to 10 wt. % of the aldehyde, or from 0.3 wt. % to 8 wt. % of the aldehyde based on the liquid weight of the solution.

If the aldehyde is present in the initial feed solution in an amount greater than 15 wt. %, or greater than the desired amount within the ranges set forth above, the initial feed solution may be diluted with solvent to obtain the desired concentration of aldehyde. For example, if the aldehyde is 3-hydroxypropionaldehyde in an aqueous solution at a concentration of greater than 15 wt. %, the initial feed solution may be diluted to the desired concentration by the addition of an aqueous liquid, e.g. water or aqueous 1,3-propanediol. It may be desirable to dilute the initial feed solution to reduce the concentration of the aldehyde in order to reduce the likelihood of formation of undesirable byproducts.

Alternately, a higher aldehyde concentration may be used as feed to a backmixed reactor, such that reaction products serve to dilute the aldehyde concentration below 15 wt % upon mixing of the feed solution with the reactor contents.

The initial feed solution containing the aldehyde may have a pH, or may be adjusted to a pH, at which the aldehyde may be inhibited from converting to undesirable byproducts, for example, acetals, or aldol condensation products. The initial feed solution containing the aldehyde may also have a pH, or may be adjusted to a pH, at which the aldehyde may be efficiently converted in a hydrogenation reaction. Preferably the initial feed solution containing the aldehyde may have a pH, or may be adjusted to a pH, at which the aldehyde may be efficiently converted in a hydrogenation reaction and at which the aldehyde may be inhibited from converting to undesirable byproducts, and at which the catalyst is not harmed by exposure to acid or base components. In one embodiment, the initial feed solution containing the aldehyde may have a pH, or may be adjusted to a pH, of at least 2.0, at least 3.0, or at least 4.0 In one embodiment, the initial feed solution containing the aldehyde may have a pH, or may be adjusted to have a pH, of at most 7.0, at most 6.5, at most 6.0, or at most 5.5. In one embodiment, the initial feed solution may have a pH, or may be adjusted to have a pH, of from 2.0 to 7.0, from 3.0 to 6.5, from 4.0 to 6.0, or from 4.0 to 5.5.

In an embodiment, the feed is a solution comprising an aldehyde, where the feed may comprise the product of an oxirane hydroformylation reaction or an aqueous extract of the product of an oxirane hydroformylation reaction. The oxirane hydroformylation reaction product may be formed by reacting an oxirane with syngas in a solvent in the presence of a hydroformylation catalyst, for example a cobalt or a rhodium based hydroformylation catalyst. The oxirane may be, for example, ethylene oxide. The solvent may be, for example, an alcohol or an ether of the formula

R₂—O—R¹

in which R₁ is hydrogen or C₁₋₂₀ linear, branched, cyclic, or aromatic hydrocarbyl or mono- or polyalkylene oxide. Preferred hydroformylation solvents include, for example, methyl-t-butyl ether, ethyl-t-butyl ether, diethyl ether, phenylisobutyl ether, ethoxyethyl ether, diphenyl ether, phenylisobutyl ether, ethoxyethyl ether, and diisopropyl ether. Blends of solvents such as tetrahydrofuran/toluene, tetrahydrofuran/heptane, and t-butylalcohol/hexane may also be used as the hydroformylation solvent. The syngas (i.e. synthesis gas) may comprise a mixture of H₂ and carbon monoxide having an H₂:CO ratio of at least 0.5:1 or at least 1:1 and at most 10:1 or 5:1. The syngas may be obtained from a commercially available source, or may be derived, for example, from a conventional methane steam reforming process.

In an embodiment, the feed may be an aqueous extract of an oxirane hydroformylation reaction mixture. The aqueous extractant used to extract the oxirane hydroformylation reaction mixture may be water, and an optional miscibilizing agent. In an embodiment, the amount of water used to extract the oxirane hydroformylation reaction mixture may generally be an amount sufficient to provide a water:reaction mixture volume ratio of from 1:1 to 1:20, or from 1:5 to 1:15. In an embodiment, the aqueous extraction may be carried out at a temperature of from 25° C. to 55° C. In an embodiment, the aqueous extraction may be carried out under 50 psig to 200 psig carbon monoxide partial pressure to maximize retention of hydroformylation catalyst in the hydroformylation reaction mixture and minimize extraction of the hydroformylation catalyst into the aqueous extractant.

The feed may be an aqueous extract of an ethylene oxide hydroformylation reaction mixture, where the feed comprises 3-hydroxypropionaldehyde. The ethylene oxide hydroformylation reaction mixture may be formed by hydroformylating ethylene oxide with syngas in a methyl-t-butyl ether solvent in the presence of a cobalt carbonyl or rhodium carbonyl catalyst to produce 3-hydroxypropionaldehyde. The feed may be produced by extracting the ethylene oxide hydroformylation reaction mixture with water or an aqueous solution. In an embodiment the feed is extracted with water or an aqueous solution under a carbon monoxide pressure of from 250 kPa to 1 MPa to minimize extraction of the hydroformylation catalyst into the aqueous extractant.

Where the aldehyde is to be hydrogenated in at least two stages, the feed comprising an aldehyde is contacted with hydrogen and a catalyst to hydrogenate the aldehyde in the feed at one or more temperatures up to about 90° C. in the presence of carbon monoxide, and then the hydrogenation is continued by contacting the feed, the catalyst, and hydrogen, optionally in the presence of carbon monoxide, at a one or more temperatures of at least about 120° C. In one embodiment, the hydrogenation of the aldehyde in the feed at one or more temperatures of up to about 90° C. may be conducted at a temperature of at least 40° C., or at least 50° C., or at least 60° C.; or at most 80° C., or at most 75° C., or at most 70° C. The hydrogenation of the aldehyde in the feed at one or more temperatures of up to 90° C. may be conducted at a temperature of from about 20° C. to about 85° C., or from about 30° C. to about 80° C., or from 40° C. to 75° C. In one embodiment the initial hydrogenation is conducted at a temperature of from 50° C. to 70° C.

The feed may be contacted with the catalyst and hydrogen to hydrogenate the aldehyde at one or more temperatures of up to 90° C. in the presence of carbon monoxide for a period effective to hydrogenate a substantial quantity of the aldehyde and insufficient for the carbon monoxide to completely inactivate the catalyst. The feed may be contacted with the catalyst and hydrogen at one or more temperatures of up to 90° C. in the presence of carbon monoxide for a period of at least 10 minutes, or at least about 15 minutes, or at least about 30 minutes, or at least about 1 hour. The feed may be contacted with the catalyst and hydrogen at one or more temperatures of up to 90° C. in the presence of carbon monoxide for a period of from 10 minutes to 5 hours, or from 15 minutes to 4 hours, or from 30 minutes to 3 hours, or from 1 hour to 2 hours.

The period of time at which the feed is contacted with the catalyst and hydrogen in the presence of carbon monoxide at one or more temperatures of up to 90° C. should be sufficient to permit hydrogenation of a substantial quantity of the aldehyde. The hydrogenation may be conducted at temperatures up to 90° C. in the presence of carbon monoxide until at least about 40%, or at least about 50%, or at least about 60%, or at least about 70%, or at least about 80%, or at least about 90% of the aldehyde has been converted.

The hydrogenation is subsequently continued by contacting the feed containing the aldehyde with hydrogen at one or more temperatures of at least about 120° C. The hydrogenation may be continued at a temperature of at least 130° C., or at least 140° C.; and may be continued at a temperature of at most 180° C., or at most 170° C., or at most 160° C.; and may be continued at a temperature of from 120° C. to 180° C., or from 130° C. to 170° C., or from 140° C. to 160° C.

The hydrogenation at one or more temperatures of at least 120° C. may be conducted for a time period effective to hydrogenate at least a majority of the aldehyde and to restore a significant amount of hydrogenation activity to the catalyst. The hydrogenation at one or more temperatures of at least 120° C. may be conducted for a time period of at least 10 minutes, or at least about 15 minutes, or at least about 30 minutes, or at least about 1 hour. The hydrogenation at one or more temperatures of at least 120° C. may be conducted for a time period of from 10 minutes to 5 hours, or from 15 minutes to 4 hours, or from 30 minutes to 3 hours, or from 1 hour to 2 hours. The hydrogenation may be conducted in a continuous process, with adjustment of the flow rate of the aqueous extractant mixture containing aldehyde passed into the hydrogenation reactor, so that the desired extent of aldehyde hydrogenation and/or catalyst reactivation is obtained.

The period of time at which the hydrogenation at one or more temperatures of at least 120° C. is conducted should be sufficient to permit hydrogenation of at least a majority, and preferably substantially all, of the aldehyde. In an embodiment of the process of the present invention, the hydrogenation at one or more temperatures of at least 120° C. converts additional aldehyde in the feed after conversion of aldehyde in the feed by hydrogenation at one or more temperatures of at most 90° C. In an embodiment of the process of the present invention, the hydrogenation at one or more temperatures of at least 120° C. effects conversion of acetal byproducts into the hydrogenation product and the aldehyde, and further hydrogenates the aldehyde reverted from the acetal into the hydrogenation product. The hydrogenation may be conducted at one or more temperatures of at least 120° C. until at least about 70%, or at least about 80%, or at least about 90%, or at least about 95%, or at least about 99% of the aldehyde has been converted, where the total amount of the aldehyde converted after contact of the feed, catalyst, and hydrogen at a temperature of at least 120° C. is greater than the total amount of aldehyde converted before contact of the feed, catalyst, and hydrogen at a temperature of at least 120° C.

The hydrogenation at one or more temperatures of at least 120° C. may also be conducted for a period of time or contact time with the catalyst, effective to reverse at least a portion of carbon monoxide poisoning of the Group VIII metal of the hydrogenation catalyst. The hydrogenation at one or more temperatures of at least 120° C. may be conducted for a period of time until the hydrogenation activity of the catalyst is at least 70%, or at least 80%, or at least 90%, or at least 95% of the initial hydrogenation activity of the catalyst, where the “hydrogenation activity” of the catalyst is measured by the amount of aldehyde hydrogenated at 60° C. and a hydrogen pressure of 1000 psi in the presence of the catalyst and in the absence of carbon monoxide for a time period of 1 hour, and the “initial hydrogenation activity” is the hydrogenation activity of the catalyst (freshly prepared) prior to hydrogenating the aldehyde in the feed in the presence of carbon monoxide.

Where the aldehyde is to be hydrogenated with a Group VIII metal catalyst complexed with carbon monoxide at a temperature of at least 120° C., the hydrogenation conditions may be the same as described above with respect to hydrogenation at a temperature of at least 120° C.

The catalyst is a hydrogenation catalyst containing a Group VIII metal. In one embodiment, the Group VIII metal may be nickel, cobalt, ruthenium, platinum, palladium, or mixtures thereof. The catalyst may include other metals, for example, copper, zinc, and chromium, and these metals may be alloyed with the Group VIII metal. Such other metals may act as promoters. If other metals are included in the catalyst, the Group VIII metal:other metals ratio, based on weight of the metals, may be at least 2:1, or at least 3:1, or at least 5:1, or at least 10:1. In an embodiment, the catalyst may be complexed with carbon monoxide.

In an embodiment of the process of the present invention, the catalyst may be a particulate, slurry, and/or bulk metal catalyst that may be dispersed as a slurry in the feed. The particulate, slurry, and/or bulk metal catalyst may contain any proportion of Group VIII metal and/or a Group VIII metal compound, including at least 0.1 wt. %, or at least 5 wt. %, or at least 50 wt. %, or at least 75 wt. %, or at least 90 wt. % of a Group VIII metal. The particulate, slurry, and/or bulk metal catalyst may consist essentially of a Group VIII metal and/or a Group VIII metal compound. A slurry catalyst useful in the process of the present invention may be a Raney nickel or a Raney cobalt catalyst.

The particulate, slurry, and/or bulk metal catalyst may be finely divided. The particulate, slurry, and/or bulk metal catalyst may have a particle size of less than 60 micrometers, or less than 50 micrometers, or less than 30 micrometers, or less than 20 micrometers, or less than 10 micrometers, or less than 5 micrometers, or less than 1 micrometer. A finely divided particulate, slurry, and/or bulk metal catalyst may be desirable to 1) aid in dispersion of the catalyst in the feed; 2) to increase selectivity of the hydrogenation to the desired product relative to fixed bed catalysts; 3) to increase catalyst life relative to fixed bed catalysts; 4) to enable high reaction rates; 5) to enable the catalyst to flow with the feed for treatment at temperatures of at most 90° C. then for treatment at temperatures of at least 120° C.; 6) for ease of reuse of the catalyst; and 7) to permit increased quantities of the aldehyde to be present in the feed without an increase in undesirable byproducts relative to fixed bed catalysts.

The particulate, slurry, and/or bulk metal catalyst may be comprised of a Group VIII metal and/or a Group VIII metal compound on a support. The support may be a carrier that is inert to conditions at which the hydrogenation is effected. Suitable inert carriers may be composed of a clay, a ceramic, or may be based on an inorganic carbide, or oxide, or carbon. For example, the support may be based on oxides of Group 2-6 and 12-14 metals and mixtures thereof, e.g. ZnO, titania, alumina, zirconia, silica, and/or zeolites. The support may be resistant to an aqueous acidic medium. The Group VIII metal and/or Group VIII metal compound of the supported particulate, slurry, and/or bulk metal catalyst may comprise at least 0.1 wt. %, or at least 5 wt. %, or at least 20 wt. %, or at least 30 wt. %, or at least 50 wt. %, or at least 60 wt. %, or at least 75 wt. %, or at least 90 wt. %, or at least 95 wt. % of the total weight of the support and the catalytic metals and/or metal compounds of the catalyst.

The particulate, slurry, and/or bulk metal catalyst based on a support may be finely divided so that the catalyst may be dispersed in the feed. The particulate, slurry, and/or bulk metal catalyst based on a support may be a fine powder. In an embodiment, the particulate, slurry, and/or bulk metal catalyst based on a support may be formed by crushing a support material having a Group VIII metal thereon into a finely divided material. In another embodiment, the particulate, slurry, and/or bulk metal catalyst may be formed by depositing a Group VIII metal onto a finely divided support material according to methods known in the art.

In an embodiment of the process of the present invention, the catalyst may be a mobile catalyst formed of a slurry, particulate, and/or bulk metal catalyst. The mobile catalyst may be dispersed in the feed for contact with hydrogen and the aldehyde in the feed. In an embodiment, the mobile catalyst, when dispersed in the feed, may comprise up to 30 wt. %; or at most 20 wt. %, or at most 15 wt. %, or at most 10 wt. %, or at most 5 wt. %, or at most 2.5 wt. % of the combined weight of the mobile catalyst and the feed. In an embodiment, the mobile catalyst, when dispersed in the feed, may comprise at least 0.1 wt. %, or at least 0.5 wt. %, or at least 1 wt. % or at least 1.5 wt. % of the combined weight of the mobile catalyst and the feed. In an embodiment, the mobile catalyst, when dispersed in the feed, may comprise from 0.1 wt. % to 10 wt. % of the combined weight of the mobile catalyst and the feed, or from 0.5 wt. % to 5 wt. % of the combined weight of the mobile catalyst and the feed, or from 1 wt. % to 2.5 wt. % of the combined weight of the mobile catalyst and the feed.

In another embodiment of the process of the present invention, the catalyst may be a fixed bed catalyst. The fixed bed catalyst may be comprised of a Group VIII metal and/or Group VIII metal compound on a support, where the catalyst is of sufficient particle size for use in a fixed-bed operation, which generally may be from about 10 micrometers to about 3 millimeters. Materials useful for forming the support for the fixed-bed type catalyst may be those described above for supported particulate, slurry, or bulk metal catalysts. The Group VIII metal and/or Group VIII metal compound of the fixed bed supported Group VIII metal catalyst may comprise at least 0.1 wt. %, or at least 0.5 wt. %, or at least 1 wt. %, or at least 2.5 wt. %, or at least 5 wt. %, or at least 10 wt. %, of the total weight of the support and the catalytic metals of the catalyst, and may comprise at most 95 wt. %, or at most 50 wt. %, or at most 30 wt. %, or at most 25 wt. %, or at most 20 wt. %, or at most 15 wt. % of the total weight of the support and the catalytic metals and/or metal compounds of the catalyst.

At any point in time in a continuous process in which a mass of feed is contacted with a mass of catalyst, a fixed bed catalyst, when in contact with the feed, may comprise up to 80 wt. %, up to 50 wt. %, up to 10 wt. % or up to 2 wt. % of the combined weight of the fixed bed catalyst and the feed. The fixed bed catalyst, when in contact with the feed, may comprise at least 0.5 wt. %, at least 10 wt. %, at least 25 wt. %, at least 50 wt. %, or at least 80 wt. % of the combined weight of the fixed bed catalyst and the feed. In an embodiment, the fixed bed catalyst, when in contact with the feed, may comprise from 1 wt. % to 80 wt. %, or from 5 wt. % to 50 wt. %, or from 10 wt. % to 35 wt. % of the combined weight of the fixed bed catalyst and the feed.

Group VIII metal catalysts useful in the process of the present invention, including particulate, slurry, bulk metal, and fixed-bed catalysts, may be formed according to conventional methods known in the art. Many such Group VIII metal catalysts are available commercially, from, for example, Criterion Corporation, Inc.

In the processes of the present invention, hydrogen is provided from a hydrogen source for contact with the feed and the catalyst to hydrogenate the aldehyde in the feed. In an embodiment, hydrogen may be provided in an amount in excess of the amount necessary to convert all of the aldehyde in the feed. In an embodiment, hydrogen is provided at a hydrogen partial pressure of at least 1 MPa, or at least 2 MPa, or at least 4 MPa, or at least 5 MPa. In an embodiment, hydrogen is provided at a hydrogen partial pressure of at most 15 MPa, or at most 12 MPa, or at most 10 MPa. In an embodiment, hydrogen is provided at a hydrogen partial pressure of from 1 MPa to 15 MPa, or from 2 MPa to 12 MPa, or from 4 MPa to 10 MPa.

In a process of the present invention, carbon monoxide may be present when the feed comprising an aldehyde is contacted with hydrogen and the Group VIII metal catalyst at a temperature up to 90° C. In an embodiment, carbon monoxide may be present at a carbon monoxide partial pressure of at least 5 kPa, or at least 60 kPa, or at least 100 kPa, or at least 200 kPa, or at least 750 kPa when the feed is contacted with the catalyst and with hydrogen at one or more temperatures up to 90° C. In an embodiment, carbon monoxide may be present at a carbon monoxide partial pressure of at least 5 kPa and at most 200 kPa, or at most 150 kPa, or at most 100 kPa when the feed is contacted with the catalyst and with hydrogen to hydrogenate the aldehyde in the feed at one or more temperatures up to 90° C. to inhibit rapid carbon monoxide poisoning of the catalyst. In an embodiment of the process of the invention, carbon monoxide may be present at a carbon monoxide partial pressure of at least 5 kPa, or at least 60 kPa, or at least 100 kPa, or at least 200 kPa, or at least 750 kPa when the feed is contacted with the catalyst and with hydrogen at one or more temperatures of at least 120° C. In an embodiment, when the feed and catalyst are contacted with hydrogen at one or more temperatures of at least 120° C. carbon monoxide may be present at a carbon monoxide partial pressure of at least 80% of the carbon monoxide partial pressure utilized when contacting the feed and catalyst with hydrogen at one or more temperatures up to 90° C. prior to contacting the feed and catalyst with hydrogen at one or more temperatures of at least 120° C. In an embodiment, the feed and catalyst may be contacted with hydrogen at one or more temperatures of at least 120° C. in the absence of a carbon monoxide partial pressure. In an embodiment of the process of the present invention, carbon monoxide may be present in the feed or in the hydrogen source.

The hydrogenation of the processes of the present invention may be carried out in conventional hydrogenation reactors, and may be a continuous process or a batch process. For example, a stirred reactor, flow reactor, or an ebullating bed reactor may be used to hydrogenate the aldehyde when a mobile catalyst such as a suspension or a slurry catalyst is used. A fixed bed hydrogenation reactor may be used to hydrogenate the aldehyde when a fixed bed catalyst is used.

In an embodiment, the process of the present invention may be a continuous process. In an embodiment, the process is a continuous process in which the feed is introduced and passed through the hydrogenation reactor or reactors at a liquid hourly space velocity (LHSV) of at least 0.1 h⁻¹, or at least 0.2 h⁻¹, or at least 0.4 h⁻¹, and at most 10 h⁻¹, or at most 7.5 h⁻¹, or at most 5 h⁻¹. The process may be a continuous process in which the feed is introduced and passed through the hydrogenation reactor or reactors at a LHSV of from 0.1 h⁻¹ to 10 h⁻¹, or from 0.2 h⁻¹ to 7.5 h⁻¹, or from 0.4 h⁻¹ to 5 h⁻¹.

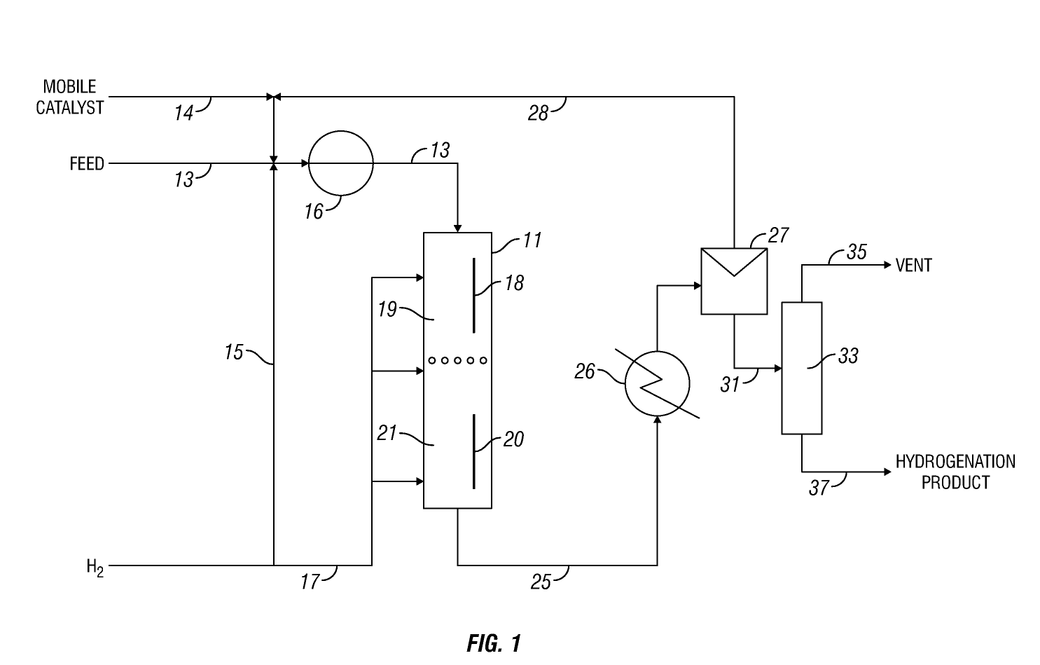

In an embodiment of the process of the present invention, as shown in FIG. 1, the process of the invention may be effected in a system having a hydrogenation reactor 11. The catalyst used may be a mobile catalyst comprising a Group VIII metal, such as a slurry or bulk metal catalyst, capable of flowing with the feed through the reactor 11. In an embodiment, the catalyst may be complexed with carbon monoxide. A feed input line 13 may direct a feed comprising an aldehyde into the reactor 11. The feed may be a hydroformylation reaction mixture or an aqueous extract of a hydroformylation reaction mixture, where the hydroformylation reaction mixture or aqueous extract thereof may be under carbon monoxide partial pressure of at least 5 kPa. The feed may flow upwardly through the reactor 11, or, as shown, may flow downwardly through the reactor 11. Hydrogen may be mixed with the feed prior to entering the reactor though line 15 and/or may be directly added to the reactor through line(s) 17. The hydrogen may be mixed with carbon monoxide, for example, as syngas. Hydrogen may be thoroughly dispersed in the feed prior to the feed and hydrogen entering the reactor, e.g. by static mixers 16.

In an embodiment, a mobile Group VIII metal containing catalyst is present in the reactor 11, and may be mixed with the feed and hydrogen entering the reactor to disperse the catalyst in the feed and ensure thorough contact of the catalyst, hydrogen, and aldehyde in the feed. The mobile catalyst may be mixed in the reactor with the feed by the flow of the feed, by stirring, or by other known means for dispersing a slurry type catalyst in a hydrogenation mixture. In another embodiment, the mobile catalyst may be added to and mixed with the feed prior to entering the reactor. The mobile catalyst may be added to the feed through line 14 and mixed with the feed, and hydrogen if hydrogen is added to the feed through line 15, in mixer 16. The mobile catalyst may be complexed with carbon monoxide.

In an embodiment, the reactor may have a single reaction zone 19 and 21. The reactor having a single reaction zone may be equipped with heating and cooling elements 18 and 20 in such a way that a reaction temperature can be established and maintained in the reaction zone of at least 120° C., or from 120° C. to 180° C., or from 130° C. to 170° C., or from 140° C. to 160° C. The reaction zone 19 and 21 may have a substantially constant temperature or may have a temperature gradient therein. A Group VIII metal catalyst complexed with carbon monoxide may be located in the reaction zone, where the carbon monoxide complexed with the catalyst may be disproportionated from the catalyst upon heating to a temperature of at least 120° C. Additional reaction zones may be included in the reactor located downstream of the reaction zone 19 and 21 having a higher temperature than the reaction zone for the purpose of reverting byproducts such as acetals to the desired hydrogenation product.

The mixture of feed and hydrogen may be contacted with the catalyst complexed with carbon monoxide in the single reaction zone 19 and 21 to convert the aldehyde at a temperature of at least 120° C. and to disproportionate the carbon monoxide complexed with the catalyst to remove the carbon monoxide from the catalyst. The mixture of feed and hydrogen, and optionally catalyst if the catalyst is a mobile catalyst, may flow through the reaction zone 19 and 21. Additional hydrogen may be added as the mixture flows through the reactor 11, if needed, through hydrogen inlets 17 in the reactor 11.

The hydrogenation product mixture may be removed from the reaction zone through outlet 25. The hydrogenation product mixture may be cooled by passing the hydrogenation product mixture exiting the reactor through a heat exchanger 26. Mobile catalyst may be removed from the cooled hydrogenation product mixture by separating the catalyst from the hydrogenation product mixture using a conventional solid/liquid separation means, e.g., by filtering the catalyst through a filter 27, or centrifugation. The catalyst may be recycled for re-use in the reactor 11 through line 28. If desired, a portion of the catalyst for re-use may be removed and replaced by fresh catalyst.

The hydrogenation product mixture may be collected from the filter 27/separation means via line 31, and the hydrogenation product may be separated from vent gases in separator 33. The vent gases may be removed from the separator 33 through line 35 and the hydrogenation product may be collected from the separator through line 37.

In an embodiment, the reactor may have at least two reaction zones 19 and 21 having separate and distinct temperature profiles. The reactor 11 may be equipped with heating or cooling elements 18 and 20 in such a way that a reaction temperature can be established and maintained in a first reaction zone 19 of up to at most 90° C., or from 40° C. to 80° C., or from 50° C. to 75° C., or from 50° C. to 60° C.; and a reaction temperature can be established and maintained in a second reaction zone 21 of at least 120° C., or from 120° C. to 180° C., or from 130° C. to 170° C., or from 140° C. to 160° C. The reaction zones 19 and 21 may have a substantially constant temperature or may have a temperature gradient therein. Additional reaction zones may be included in the reactor located downstream of the second reaction zone and having a higher temperature than the second reaction zone for the purpose of reverting byproducts such as acetals to the desired hydrogenation product.

A mixture of feed, hydrogen, catalyst, and carbon monoxide may be first contacted in the first reaction zone 19 to convert the aldehyde at a temperature of at most 90° C. The mixture of feed, hydrogen, and catalyst may flow through the first reaction zone 19 and into the second reaction zone 21, where the conversion of the aldehyde may be continued at a temperature of at least 120° C. Additional hydrogen may be added as the mixture flows through the reactor 11, if needed, through hydrogen inlets 17 in the reactor 11.

The hydrogenation product mixture may be removed from the second reaction zone 21 of reactor 11 through outlet 25. The hydrogenation product mixture may be cooled by passing the hydrogenation product mixture exiting the reactor through a heat exchanger 26. Catalyst may be removed from the cooled hydrogenation product mixture by separating the catalyst from the hydrogenation product mixture using a conventional solid/liquid separation means, e.g. by filtering the catalyst through a filter 27, or centrifugation. The catalyst may be recycled for re-use in the reactor 11 through line 28. If desired, a portion of the catalyst for re-use may be removed and replaced by fresh catalyst.

The hydrogenation product mixture may be collected from the filter 27/separation means via line 31, and the hydrogenation product may be separated from vent gases in separator 33. The vent gases may be removed from the separator 33 through line 35 and the hydrogenation product may be collected from the separator 33 through line 37.

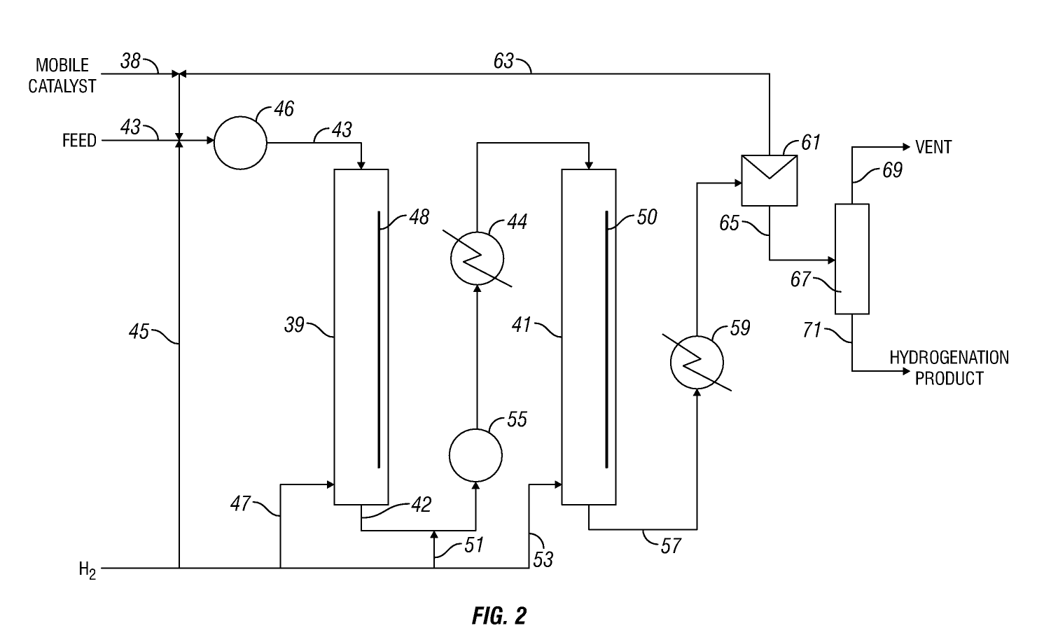

In an alternative embodiment as shown in FIG. 2, first reaction and second reaction zones comprise separate hydrogenation reactors 39 and 41 each having one or more heating elements 48 and 50 for heating and maintaining the reactors 39 and 41 at desired temperatures, where the first hydrogenation reactor 39 may be maintained and operated at a temperature of at most 90° C., and the second hydrogenation reactor 41 may be maintained and operated at a temperature of at least 120° C. The catalyst used in the multiple reactor system may be a mobile catalyst comprising a Group VIII metal, such as a slurry or bulk metal catalyst, capable of flowing with the feed through the reactors 39 and 41. A feed input line 43 may direct a feed comprising an aldehyde into the first hydrogenation reactor 39. The feed may be a hydroformylation reaction mixture or an aqueous extract of a hydroformylation reaction mixture, where the hydroformylation reaction mixture or aqueous extract thereof may be under carbon monoxide partial pressure of at least 25 kPa. The feed may flow upwardly through the first hydrogenation reactor 39, or, as shown, may flow downwardly through the reactor 39. Hydrogen may be mixed with the feed prior to entering the reactor though line 45 or may be directly added to the reactor through line 47. The hydrogen may be mixed with carbon monoxide, for example, as syngas. Hydrogen may be thoroughly dispersed in the feed prior to the feed and hydrogen entering the reactor, e.g. by static mixers 46.

In an embodiment, the mobile Group VIII metal catalyst may be added to and mixed with the feed prior to entering the first hydrogenation reactor 39 through line 38. The mobile catalyst may be mixed with the feed, and hydrogen if hydrogen is added to the feed through line 45, in mixer 46.

The reaction temperature may be established and maintained in the first hydrogenation reactor 39 at a temperature of up to at most 90° C., or from 40° C. to 80° C., or from 50° C. to 75° C., or from 50° C. to 60° C. The first hydrogenation reactor 39 may include heating means 48 to establish and maintain a reaction temperature in the reactor 39. The reaction temperature may be held constant through the first hydrogenation reactor 39 or a temperature gradient may be established in the first hydrogenation reactor 39. In one embodiment, a temperature gradient is established in the first hydrogenation reactor 39 such that the temperature increases as the reaction mixture of feed and catalyst flow through the reactor.

The feed and catalyst may exit the first hydrogenation reactor 39 through line 42 and proceed to the second hydrogenation reactor 41. In an embodiment, the feed and catalyst may be heated by a heat exchanger 44 between the first hydrogenation reactor 39 and the second hydrogenation reactor 41 to raise the temperature of the feed and the catalyst to at least 120° C. Hydrogen may be mixed with the feed and catalyst prior to entering the second hydrogenation reactor 41 though line 51 or may be directly added to the reactor through line 53. The hydrogen may be mixed with carbon monoxide, for example, as syngas. Hydrogen may be thoroughly dispersed in the feed and catalyst prior to the feed and hydrogen entering the reactor, e.g. by static mixer 55.

The feed and catalyst may flow upwardly through the second hydrogenation reactor 41, or, as shown, may flow downwardly through the reactor 41. As noted above, hydrogen may be mixed with the feed and catalyst prior to entering the second hydrogenation reactor 41, or the hydrogen may be mixed with the feed and catalyst in the reactor 41. Hydrogen may be passed through the second hydrogenation reactor 41 in a flow countercurrent to the flow of the feed and catalyst through the reactor 41 or co-current with the flow of the feed and catalyst through the reactor 41.

The reaction temperature may be established and maintained in the second hydrogenation reactor 41 at a temperature of up to at least 120° C., or from 120° C. to 180° C., or from 125° C. to 175° C., or from 130° C. to 170° C. The second hydrogenation reactor 41 may include heating means 50 to establish and maintain a reaction temperature in the reactor 41. The reaction temperature may be held constant through the second hydrogenation reactor 41 or a temperature gradient may be established in the second hydrogenation reactor 41. In one embodiment, a temperature gradient is established in the second hydrogenation reactor 41 such that the temperature increases as the reaction mixture of feed and catalyst flow through the reactor 41.

Additional hydrogenation reactors may be included downstream of the second hydrogenation reactor 41 and having an equivalent or higher temperature than the second hydrogenation reactor 41 for the purpose of reverting byproducts such as acetals to the desired hydrogenation product.

The hydrogenation product mixture may be removed from the second hydrogenation reactor 41 through outlet 57. The hydrogenation product mixture may be cooled by passing the hydrogenation product mixture exiting the reactor 41 through a heat exchanger 59. Catalyst may be removed from the cooled hydrogenation product mixture by separating the catalyst from the hydrogenation product mixture using a conventional solid/liquid separation means, e.g. by filtering the catalyst through a filter 61, or centrifugation. The catalyst may be recycled for re-use in the reactors 39 and 41 through line 63. If desired, a portion of the catalyst for re-use may be removed and replaced by fresh catalyst.

The hydrogenation product mixture may be collected from the filter 61/separation means via line 65, and the hydrogenation product may be separated from vent gases in separator 67. The vent gases may be removed from the separator 67 through line 69 and the hydrogenation product may be collected from the separator 67 through line 71.

In another alternative embodiment, the hydrogenation reactor comprises only one reaction zone, where the hydrogenation reactor is equipped with one or more heating elements for heating and maintaining the reactor at a temperature of up to 90° C. and for further heating and maintaining the reactor at a temperature of at least 120° C. The catalyst in the one reaction zone may be a mobile Group VIII metal catalyst, such as a slurry catalyst or a bulk metal catalyst, or the catalyst may be a fixed bed Group VIII metal catalyst. The feed comprising an aldehyde and hydrogen may be fed to the reactor in the same manner described above. The reactor may initially be established and maintained at a temperature of up to 90° C., or from 40° C. to 80° C., or from 50° C. to 75° C., or from 50° C. to 60° C. The feed, catalyst, hydrogen, and carbon monoxide may be contacted at the initial temperature in the reactor until at least 40%, or at least 50%, or at least 60%, or at least 70%, or at least 80%, or at least 90% of the aldehyde has been converted—typically at least 30 minutes, or at least 45 minutes, or at least 1 hour. The reactor temperature may then be increased to be established and maintained at a temperature of at least 120° C., or from 120° C. to 180° C., or from 130° C. to 170° C., or from 140° C. to 160° C. until at least 70%, or at least 80%, or at least 90%, or at least 95%, or at least 99% of the aldehyde has been converted and/or until the activity of the catalyst is at least 70%, or at least 80%, or at least 90%, or at least 95% of the initial activity of the catalyst—typically at least 30 minutes, or at least 45 minutes, or at least 1 hour.

The hydrogenation product may then be separated from the catalyst in the one reaction zone hydrogenation reactor. In an embodiment, the hydrogenation product may be removed from the hydrogenation reactor through an outlet line. If the hydrogenation catalyst is a mobile catalyst, for example a slurry catalyst, the hydrogenation product may be passed through a separator, for example a filter or a centrifuge, for separating the catalyst from the hydrogenation product. The catalyst, either a separated mobile catalyst or a fixed bed catalyst, may be reused in the reactor for further hydrogenation.

In an embodiment, a combination of reactors or reaction zones may be used to hydrogenate an aldehyde in the presence of carbon monoxide where the order of the reactors or reaction zones may be periodically reversed. A first reactor or reaction zone containing a Group VIII metal catalyst may be used initially to hydrogenate a feed containing an aldehyde in the presence of carbon monoxide at a temperature up to 90° C., where a second reactor or reaction zone containing a Group VIII metal catalyst may be used initially to hydrogenate aldehyde in a feed exiting the first reactor or reactor zone at a temperature of at least 120° C. The first reactor or reaction zone may be utilized to hydrogenate the aldehyde in the presence of carbon monoxide at a temperature of at most 90° C. for a period of time until the hydrogenation activity of the catalyst is significantly diminished due to poisoning by carbon monoxide. Upon significantly diminished catalytic activity in the first reactor or reaction zone, the order of the first reactor or reaction zone and the second reactor or reaction zone may be switched, where the second reactor or reaction zone is used to hydrogenate a feed containing an aldehyde in the presence of carbon monoxide at a temperature of at most 90° C. and the first reactor or reaction zone is then used to hydrogenate a feed exiting from the second reactor or reaction zone at a temperature of at least 120° C. Switching the order of the first and second reactors or reaction zones on a periodic basis permits the high temperature reversal of carbon monoxide poisoning of the catalysts in the reactors. In this mode, it is not necessary to transport catalyst between zones, and the invention may be applied to a fixed-bed catalyst.

The hydrogenation product, whether produced in one reaction zone/hydrogenation reactor or in multiple reaction zones/hydrogenator reactors, may be purified to produce the desired product by removal of the feed solvent and byproducts. The feed solvent and byproducts may be separated from the desired product by distillation, which may include multiple distillations to separate light ends/solvent from the desired product in a first distillation step, and to separate the desired product from heavy ends/bottoms in a second distillation step.

In an embodiment, the invention is a process for producing 1,3-propanediol. An aqueous feed may be provided that comprises 3-hydroxypropionaldehyde. The feed may be contacted with hydrogen and a catalyst comprising a Group VIII metal at a temperature of up to about 90° C., or about 30° C. to about 85° C., or about 40° C. to about 80° C. in the presence of carbon monoxide, in an embodiment under a carbon monoxide partial pressure of at least 25 kPa. The feed may be contacted with the catalyst and hydrogen in the presence of carbon monoxide at a temperature of up to 90° C. until at least 40%, or at least 50%, or at least 60%, or at least 70%, or at least 80%, or at least 90% of 3-hydroxypropionaldehyde has been converted to 1,3-propanediol—typically at least 30 minutes, or at least 45 minutes, or at least 1 hour. The feed and catalyst are subsequently contacted with hydrogen at a temperature of from about 120° C. to about 180° C. to produce a hydrogenation product mixture containing 1,3-propanediol. The feed and catalyst may be contacted with hydrogen at a temperature of from 120° C. to 180° C. until at least 70%, or at least 80%, or at least 90%, or at least 95%, or at least 99% of 3-hydroxypropionaldehyde has been converted to 1,3-propanediol. The feed and catalyst may be contacted with hydrogen at a temperature of from 120° C. to 180° C. for a period of at least 10 minutes, or least about 15 minutes, or at least about 30 minutes, or at least 45 minutes, or at least about 1 hour.

In a preferred embodiment of the process of the present invention, the aqueous feed is an aqueous extract of a hydroformylation reaction mixture containing 3-hydroxypropionaldehyde. To produce the hydroformylation reaction mixture, separate or combined streams of ethylene oxide, carbon monoxide, and hydrogen may be charged to a hydroformylation reaction vessel, which can be a pressure reaction vessel such as a bubble column or an agitated tank, operated batchwise or in a continuous manner. The feed streams may be contacted in the presence of a hydroformylation catalyst. The hydroformylation catalyst may comprise one or more transition metal species. The transition metal of the species may be one or more metals of transition group VIII of the Periodic Table, preferably cobalt, ruthenium, rhodium, palladium, platinum, osmium, and iridium, more preferably cobalt or rhodium. The transition metal species may be a carbonyl, in particular a water-insoluble cobalt or rhodium carbonyl such as Co[Co(CO)₄], Co₂(CO)₈, and Rh₆(CO)₁₆. The hydroformylation catalyst may be present in the reaction mixture in an amount in the range of from 0.01 wt. % to 1 wt. %, or from 0.05 wt. % to 0.3 wt. %, relative to the weight of the hydroformylation reaction mixture. The hydrogen and carbon monoxide may be introduced into the reaction vessel in a molar ratio in the range of 1:2 to 8:1, preferably 1:1 to 6:1, and may be introduced as syngas.

The hydroformylation reaction may be carried out under conditions effective to produce a hydroformylation reaction product mixture containing a major portion of 3-hydroxypropionaldehyde and a minor portion of acetaldehyde and 1,3-propanediol, while maintaining the level of 3-hydroxypropionaldehyde in the reaction mixture at less than 15 wt. %, preferably within the range of 5 to 10 wt. %, relative to the total weight of the reaction mixture. Generally, the cobalt-catalyzed hydroformylation reaction of ethylene oxide may be carried out at elevated temperatures less than 100° C., preferably 60° C. to 90° C., and most preferably 75° C. to 85° C., with rhodium-catalyzed hydroformylations of ethylene oxide on the order of about 10° C. higher. The hydroformylation reaction may be carried out at a pressure of from 1 to 35 MPa, preferably (for process economics) 7 to 25 MPa, with higher pressures preferred for greater selectivity.

The hydroformylation reaction mixture is carried out in a liquid solvent inert to the reactants, i.e. the solvent is not consumed during the course of the reaction. Preferred solvents for the hydroformylation reaction are discussed above relative to oxirane hydroformylation reactions in general, where the most preferred solvent is methyl-t-butyl ether.

To further enhance yields under moderate reaction conditions, the hydroformylation reaction mixture may include a catalyst promoter to accelerate the reaction rate. Preferred promoters include lipophilic phosphonium salts and lipophilic amines, which accelerate the rate of hydroformylation without imparting hydrophilicity to the active catalyst. The promoter may be present in the hydroformylation reaction mixture in an amount of from 0.01 mole to 1 mole per mole of metal component of the catalyst (e.g. cobalt or rhodium). Preferred promoters include tetrabutylphosphonium acetate and dimethyldodecyl amine.

At low concentrations, water may serve as a promoter for the formation of the desired carbonyl hydroformylation catalyst species. Optimum water levels for hydroformylation in methyl-t-butyl ether solvent may be in the range of from 1 wt. % to 2.5 wt. % relative to the total weight of the hydroformylation reaction mixture.

Following the hydroformylation reaction, the hydroformylation reaction product mixture may be cooled and passed to an extraction vessel for extraction with an aqueous solvent, preferably water and an optional miscibilizing agent. Liquid-liquid extraction of the 3-hydroxypropionaldehyde into the aqueous solvent may be effected by any suitable means, such as mixer-settlers, packed or trayed extraction columns, or rotating disk contactors. The amount of water added to the hydroformylation reaction product mixture may be such as to provide a water-mixture ratio of from 1:1 to 1:20, preferably 1:5 to 1:15, by volume. Extraction may be carried out at a temperature of from 25° C. to 55° C., with a lower temperature preferred. Extraction may be carried out under a 0.5 MPa to 5 MPa carbon monoxide partial pressure to minimize extraction of the hydroformylation catalyst into the aqueous phase.

The aqueous 3-hydroxypropionaldehyde solution generated from the liquid-liquid water extraction may contain from 4 wt. % to 60 wt. % 3-hydroxypropionaldehyde, relative to the total weight of the aqueous 3-hydroxypropionaldehyde solution. The aqueous 3-hydroxypropionaldehyde solution may be used as the feed for the hydrogenation process of the present invention, or the aqueous 3-hydroxypropionaldehyde solution may be diluted with water to produce the feed, as described generally above. The pH of the aqueous 3-hydroxypropionaldehyde solution feed or the diluted solution feed may be adjusted, as described generally above.

The feed derived from the hydroformylation reaction containing 3-hydroxypropionaldehyde may then be hydrogenated as described generally above to produce a hydrogenation product mixture containing 1,3-propanediol. Hydrogenation using a slurry catalyst comprised of at least 50 wt. % metal, particularly Raney cobalt, is preferred to provide selectivity to produce 1,3-propanediol and a high reaction rate.

1,3-propanediol may be separated from the hydrogenation product mixture by distilling water and light ends from the 1,3-propanediol, and subsequently distilling the 1,3-propanediol to separate the 1,3-propanediol from heavy ends.

### EXAMPLE 1

An experiment was conducted to determine the effect of the presence of carbon monoxide on the catalytic hydrogenation of 3-hydroxypropionaldehyde using a Group VIII metal containing catalyst.

Three 200 gram samples were prepared of an aqueous aldehyde feed containing between 2.5 and 4.5 wt. % of 3-hydroxypropionaldehyde (or “3-HPA”). The feed for the samples was derived from an aqueous extract of an ethylene oxide hydroformylation reaction mixture diluted 3.5 fold with deionized water and pH neutralized to a pH of 5.5 by the addition of 1N potassium hydroxide. Between 1.5 to 3.5 grams of finely divided Raney cobalt-chromium catalyst and an aldehyde feed sample were charged to a hydrogenation reactor. The first sample was charged with 1000 psig hydrogen gas, the second sample was charged with an initial dose of a 2:1 mixture of H₂/CO and subsequently charged with hydrogen gas to a pressure of 7 MPa to provide a CO partial pressure of 60 kPa (CO mol/kg-catalyst ratio of 3.3), and the third sample was charged with an initial dose of 2:1 mixture of H₂/CO and subsequently charged with hydrogen gas to a pressure of 7 MPa to provide a CO partial pressure of 230 kPa (CO mol/kg-catalyst ratio of 12.3). The reactor containing each sample was then heated to 60° C. with stirring at 800-1200 rpm for 1.5 hours. The resulting products of each sample were then cooled and analyzed to determine the amount of hydrogenation effected by the reaction. The results are shown in Table 1.

TABLE 1

| | CO partial | | |
|---|---|---|---|
| | pressure | CO mol/ | 3-HPA (% |
| Catalyst | (kPa) | kg-cat | converted) |
| Raney Co—Cr | 0 | 0 | 83 |
| Raney Co—Cr | 60 | 3.3 | 58 |
| Raney Co—Cr | 230 | 12.3 | 0 |

The experiment showed that increasing levels of carbon monoxide inhibited hydrogenation of 3-hydroxypropionaldehyde. The experiment also showed that at low levels of carbon monoxide some hydrogenation activity occurred.

### EXAMPLE 2

An experiment was conducted to show that a Group VIII metal hydrogenation catalyst previously exposed to carbon monoxide is relatively ineffective to hydrogenate an aldehyde even though carbon monoxide is not present in the hydrogenation reaction.

An aqueous 3-hydroxypropionaldehyde feed was prepared as described above in Example 1. 120 grams of the 3-hydroxypropionaldehyde feed and 1.6 grams of a chromium promoted Raney cobalt catalyst were charged to a reactor. A mixture of 1:1 H₂/CO syngas was added to the reactor, followed by pressurization with hydrogen gas to 7 MPa, such that the carbon monoxide was present at a partial pressure of 60 kPa. The reactor was heated to 60° C. for one hour, and a sample was taken to determine the extent of conversion of the 3-hydroxypropionaldehyde. The reactor was then vented and the feed deinventoried from the reactor via a filtered dip tube while retaining the catalyst in the reactor. A second charge of feed was then added to the reactor and hydrogen gas was added to the reactor to a pressure of 7 MPa. The reactor was again heated to 60° C., and samples were taken to determine the extent of conversion of 3-hydroxypropionaldehyde after 1 hour of reaction and after 2.5 hours of reaction. The results are shown in Table 2.

TABLE 2

| | Reaction | CO Partial | | |
|---|---|---|---|---|
| | Time | Pressure | CO mol/ | 3-HPA (% |
| Catalyst | (hours) | (kPa) | kg-cat | converted) |
| Raney Co—Cr | 1 | 60 | 4.7 | 6.4 |
| Raney Co—Cr | 1 | 0 | 0 | 13.9 |
| (previously exposed | | | | |
| to CO) | | | | |
| Raney Co—Cr | 2.5 | 0 | 0 | 31.0 |
| (previously exposed | | | | |
| to CO) | | | | |

The experiment showed that exposure of a Group VIII metal hydrogenation catalyst to carbon monoxide inhibited the hydrogenation activity of the catalyst at a temperature of 60° C. even in the subsequent absence of carbon monoxide partial pressure in the reaction atmosphere.

### EXAMPLE 3

An experiment was conducted to show that a Group VIII metal hydrogenation catalyst previously exposed to carbon monoxide is relatively effective to hydrogenate an aldehyde at a reaction temperature above 120° C., and thereafter the catalyst is relatively effective to hydrogenate an aldehyde at a temperature below 90° C.

The hydrogenation reaction of Example 2 wherein the feed was reacted at a temperature of 60° C. for 2.5 hours was continued at a temperature of 150° C. under an atmosphere of 7 MPa H₂. After 13.5 hours at 150° C. a sample was taken to determine the extent of conversion of the 3-hydroxypropionaldehyde. The reactor was then vented and the feed deinventoried from the reactor via a filtered dip tube while retaining the catalyst in the reactor. Another charge of feed as prepared in Example 2 was then added to the reactor and hydrogen gas was added to the reactor to a pressure of 7 MPa. The reactor was then heated to 60° C., and a sample was taken to determine the extent of conversion of 3-hydroxypropionaldehyde after 1 hour of reaction. The results are shown in Table 3.

TABLE 3

| | Reaction | | CO Partial | | |
|---|---|---|---|---|---|
| | Time | Reaction | Pressure | CO mol/ | 3-HPA (% |
| Catalyst | (hours) | Temperature | (kPa) | kg-cat | converted) |
| Raney Co—Cr | 13.5 | 150° C. | 0 | 0 | 97.1 |
| (previously | | | | | |
| exposed to CO) | | | | | |
| Raney Co—Cr | 1 | 60° C. | 0 | 0 | >97% |
| (previously | | | | | |
| exposed to CO, | | | | | |
| then treated at a | | | | | |
| temperature of | | | | | |
| 150° C.) | | | | | |

The experiment showed that exposure of a Group VIII metal hydrogenation catalyst poisoned with carbon monoxide is effective to hydrogenate an aldehyde at a temperature greater than 120° C. (150° C.), and that a Group VIII metal hydrogenation catalyst previously poisoned with carbon monoxide and subsequently treated at a temperature of 150° C. is effective to hydrogenate an aldehyde at a temperature lower than 90° C. (60° C.).

### EXAMPLE 4

An experiment was conducted to show that Group VIII metal hydrogenation catalysts other than Raney Co—Cr are poisoned by carbon monoxide at temperatures below 120° C. Experiments were conducted with powdered forms of commercially available platinum and ruthenium catalysts supported on a carbon support under the conditions described in Example 1. As shown in Table 4, hydrogenations conducted in the presence of carbon monoxide at 60° C. were severely inhibited relative to those conducted with no carbon monoxide present.

TABLE 4

| | CO partial | | |
|---|---|---|---|
| | pressure | CO mol/ | 3-HPA (% |
| Catalyst | (kPa) | kg-cat | converted) |
| 5% Ru/Carbon | 0 | 0 | 50 |
| 5% Ru/Carbon | 1300 | 83.5 | 0 |
| 5% Pt/Carbon | 0 | 0 | 52 |
| 5% Pt/Carbon | 230 | 8.4 | 2 |

What is claimed is:

1. A process for hydrogenating an aldehyde comprising contacting a feed comprising an aldehyde with hydrogen and with a catalyst at a temperature of at least 120° C., where the catalyst comprises a Group VIII metal, or a compound containing a Group VIII metal, and where the Group VIII metal or Group VIII metal compound is complexed with carbon monoxide.

2. The process of claim 1 wherein the feed is an aqueous feed.

3. The process of claim 1 wherein the aldehyde is 3-hydroxypropionaldehyde.

4. The process of claim 1 wherein the catalyst comprises a metal selected from the group consisting of nickel, cobalt, palladium, platinum, rhodium, iron, ruthenium, and mixtures thereof.

5. The process of claim 1 wherein the feed is contacted with the catalyst and hydrogen at a temperature of at least 120° C. until at least 70% of the aldehyde has been converted.

6. A process for hydrogenating an aldehyde in the presence of carbon monoxide, comprising:
(a) contacting a feed comprising an aldehyde with hydrogen and a catalyst comprising a Group VIII metal or a compound of a Group VIII metal at a temperature up to 90° C. in the presence of carbon monoxide; and
(b) subsequent to step (a), contacting the feed and catalyst with hydrogen at a temperature of at least 120° C. to produce a hydrogenation product.

7. The process as claimed in claim 6 wherein the carbon monoxide is present at a partial pressure of at least 1 kPa.

8. The process of claims 6 further comprising the step of separating the hydrogenation product from the catalyst and re-using the separated catalyst by contacting the separated catalyst with the feed and hydrogen to hydrogenate the aldehyde.

9. The process of claim 6 wherein the feed is an aqueous feed.

10. The process of claim 9 wherein the aqueous feed is an aqueous extract of an oxirane hydroformylation product mixture containing an aldehyde.

11. The process of claim 6 wherein the process is conducted in
at least two reaction zones where step (a) is conducted in a first reaction zone and step (b) is conducted in a second reaction zone.

12. The process of claim 6 wherein the catalyst comprises a metal or a compound thereof selected from the group consisting of nickel, cobalt, palladium, platinum, rhodium, iron, ruthenium, and mixtures thereof.

13. The process of claim 6 wherein the feed is contacted with the catalyst and hydrogen in the presence of carbon monoxide at a temperature of up to 90° C. for a period of at least 15 minutes.

14. The process of claim 6 wherein the feed is contacted with the catalyst and hydrogen in the presence of carbon monoxide at a temperature of up to 90° C. until at least 40% of the aldehyde is converted.

15. The process of claim 14 wherein the feed is contacted with the catalyst and hydrogen at a temperature of at least 120° C. until at least 70% of the aldehyde is converted, where the total amount of the aldehyde converted after contact of the feed, catalyst, and hydrogen at a temperature of at least 120° C. is greater than the total amount of aldehyde converted before contact of the feed, catalyst, and hydrogen at a temperature of at least 120° C.

16. A process for producing 1,3-propanediol, comprising:
a) providing an aqueous feed comprising 3-hydroxypropionaldehyde;
b) contacting said feed with hydrogen and a catalyst comprising a Group VIII metal or a compound thereof at a temperature of up to 90° C. in the presence of carbon monoxide; and
c) subsequent to step (b), contacting the feed and the catalyst with hydrogen at a temperature of from 120° C. to 180° C. to produce a hydrogenation product mixture containing 1,3-propanediol.

17. The process of claim 16 wherein the aqueous feed is an aqueous extract of an ethylene oxide hydroformylation product mixture containing the 3-hydroxypropionaldehyde.

18. The process of claim 17 wherein carbon monoxide is present in said ethylene oxide hydroformylation product mixture and the aqueous feed.

19. The process of claim 16 wherein the aqueous feed provided in step (a) is under a carbon monoxide partial pressure of at least 1 kPa and the feed is contacted with hydrogen and the catalyst in step (b) under a carbon monoxide pressure of at least 80% of the carbon monoxide pressure over the aqueous feed in step (a).

20. The process of claim 16 further comprising the step of separating the catalyst from the hydrogenation product mixture and re-using the catalyst by contacting the catalyst with said feed and hydrogen to hydrogenate said 3-hydroxypropionaldehyde.

21. The process of claim 16 wherein the process is conducted in at least two reaction zones where step (b) is conducted in a first reaction zone and step (c) is conducted in a second reaction zone.

22. The process of claim 16 wherein the catalyst comprises a metal or a compound thereof selected from the group consisting of nickel, cobalt, palladium, platinum, rhodium, iron, ruthenium, and mixtures thereof.

23. The process of claim 16 wherein the feed is contacted with the catalyst and hydrogen in the presence of carbon monoxide at a temperature of up to 90° C. for a period of at least 15 minutes.

24. The process of claim 16 wherein the feed is contacted with the catalyst and hydrogen in the presence of carbon monoxide in step (b) until at least 40% of the aldehyde is converted.

25. The process of claim 24 wherein the feed is contacted with the catalyst and hydrogen at a temperature of at least 120° C. until at least 70% of the aldehyde is been converted, where the total amount of the aldehyde is converted after contact of the feed, catalyst, and hydrogen at a temperature of at least 120° C. is greater than the total amount of the aldehyde converted before contact of the feed, catalyst, and hydrogen at a temperature of at least 120° C.
